(19) United States
(12) Patent Application Publication
(10) Pub. No.: US 2005/0254664 A1
(43) Pub. Date: Nov. 17, 2005

(54) Noise cancellation methodology for electronic devices

(76) Inventors: Wah Yiu Kwong, Beaverton, OR (US); Hong W. Wong, Portland, OR (US)

Correspondence Address: BLAKELY SOKOLOFF TAYLOR & ZAFMAN, 12400 WILSHIRE BOULEVARD, SEVENTH FLOOR, LOS ANGELES, CA, 90025-1030, US

(21) Appl. No.: 10/845,676

(22) Filed: May 13, 2004

(51) Int. Cl. A61F 11/06; G10K 11/16; H03B 29/00

(52) U.S. Cl. 381/71.2; 381/71.8

(57) ABSTRACT

A system for generating a noise cancellation waveform is described. Specifically, the system captures a noise waveform, executes a noise cancellation algorithm, and communicates the noise cancellation waveform to a working environment. The noise cancellation algorithm comprises a delay parameter, a reflective index, and distortion variables.

### FIELD OF THE INVENTION

The present invention pertains to the field of computer system design. More particularly, the present invention relates to a method to cancel noise in a working environment.

### BACKGROUND OF THE INVENTION

Sound may be defined by a waveform that travels through matter. For example, sound may travel through air, water, or metal. Insulating materials may absorb sound waves, thereby preventing them from penetrating the materials. Sound may also be defined by a waveform reflected from a material or a surface.

Sound is created by object vibrations. It follows that these vibrations may be detected. The human ear is an organ that detects sound waves. Sound waves are detected by a person when his eardrums are vibrated by the waves. The signals are then processed by the brain.

### BRIEF DESCRIPTION OF THE DRAWINGS

FIG. 1 is an embodiment of a system for generating a noise cancellation waveform;

FIG. 2 is an embodiment of a working environment having a mobile computer system that provides noise cancellation;

FIG. 3 is an embodiment of a flowchart for generating a noise cancellation waveform; and

FIG. 4 is an embodiment of a flowchart of a noise generation algorithm.

### DETAILED DESCRIPTION

In the following detailed description, numerous specific details are set forth in order to provide a thorough understanding of the invention. However, it will be understood by those skilled in the art that the present invention may be practiced without these specific details. In other instances, well-known methods, procedures, components and circuits have not been described in detail so as not to obscure the present invention.

Sound may be created by a variety of different sources. When a sound is undesirable, the sound is often considered a noise. For example, the buzzing of a florescent light, the background conversation at airports, the ticking of a clock, and the roar of an engine are typically considered sources of noise.

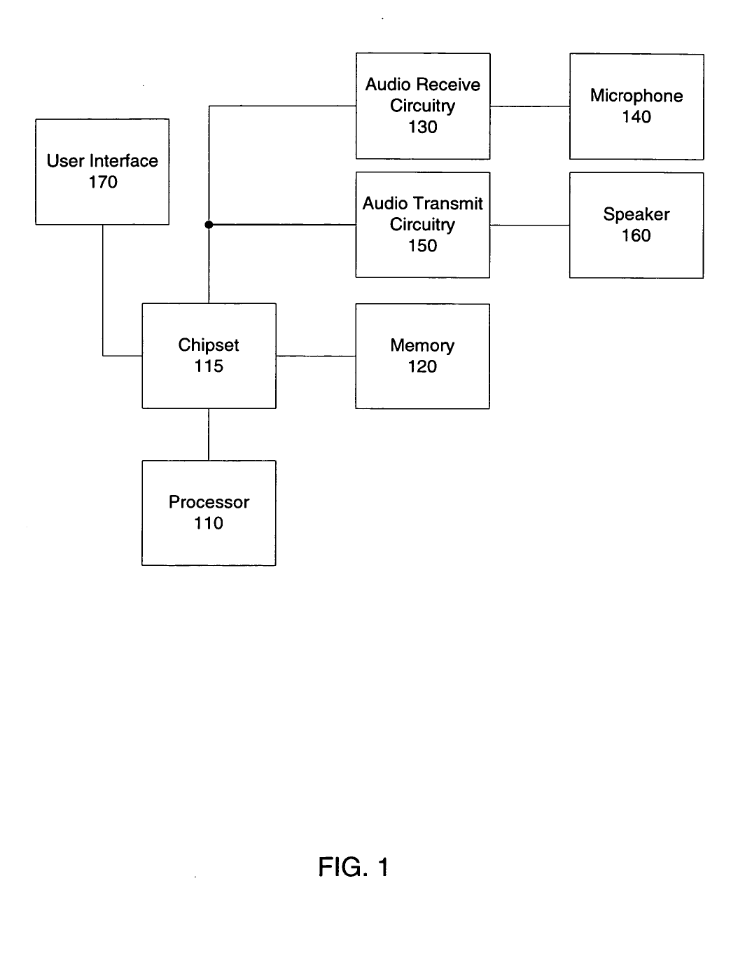

FIG. 1 depicts an embodiment of a noise cancellation system. The noise cancellation system may be part of an electronic device. The system comprises a processor 110, a chipset 115, a memory 120, an audio receive circuitry 130, a microphone 140, an audio transmit circuitry 150, a speaker 160, and a user interface 170. Processor 110 is coupled to chipset 115. Chipset 115 is coupled to memory 120, audio receive circuitry 130, audio transmit circuitry 150, and user interface 170. Audio receive circuitry 130 is coupled to microphone 140. Audio transmit circuitry 150 is coupled to speaker 160.

The noise waveform in a working environment is captured by microphone 140. The working environment may be an office or an automobile. The captured waveform may be a continuous, periodic, analog signal. The captured waveform is transmitted to the audio receive circuitry 130. The audio receive circuitry 130 may be a codec. The audio receive circuitry 130 may convert an analog signal to a digital signal. The audio receive circuitry 130 may also compress the captured waveform. The processor 110 uses the captured waveform to calculate a noise cancellation waveform or signal. The algorithm for generating the noise cancellation waveform may be stored in memory 120.

The processor 110 may execute the noise cancellation algorithm to generate the noise cancellation waveform. The noise cancellation waveform may have the same amplitude and the same frequency as the captured waveform. However, the noise cancellation waveform may be 180 degrees out of phase with respect to the captured waveform.

The user interface 170 is coupled to the processor 110 and enables an operator or user of the system to alter the noise cancellation algorithm. For example, the user may wish to modify the parameters of the noise cancellation algorithm depending on the conditions of the working environment. The user interface 170 may also enable the user to limit the frequency and the amplitude range of waveforms captured by microphone 140.

After the processor generates the noise cancellation waveform, the audio transmit circuitry 150 may amplify the noise cancellation waveform. Further, the audio transmit circuitry 150 may convert the noise cancellation waveform from a digital signal to an analog signal. Finally, the speaker 160 may communicate the noise cancellation waveform to the working environment. The noise cancellation waveform may offset the noise waveform.

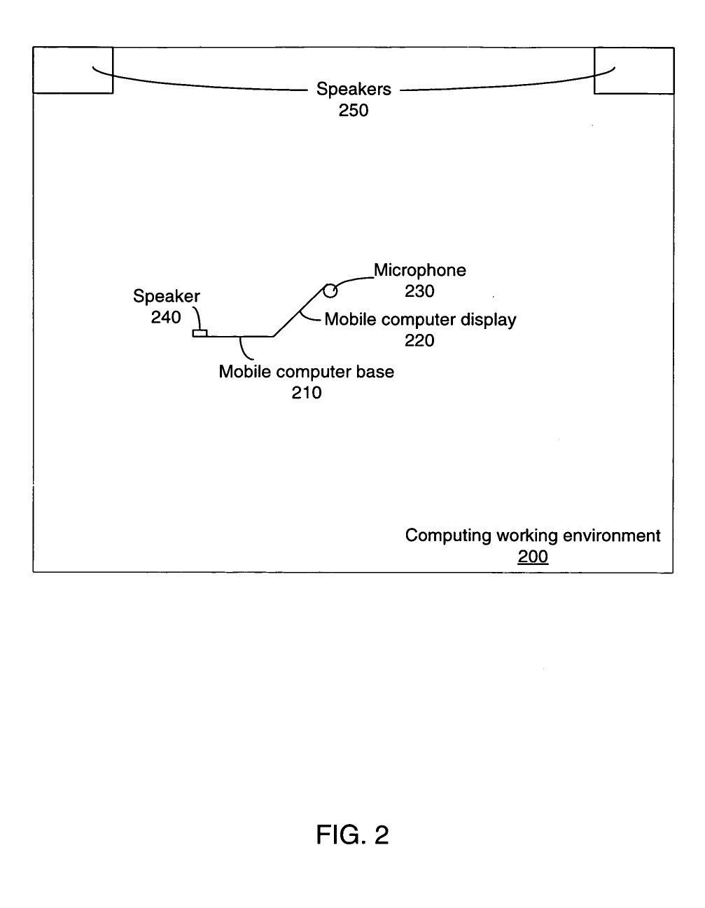

For one embodiment of the invention, the noise cancellation system of FIG. 1 may be part of a computer system. The computer system may be a mobile computer system, desktop computer system, or server computer system. FIG. 2 depicts a computing working environment 200 having a mobile computer system that provides noise cancellation. The mobile computer system comprises a computer base 210, a computer display 220, a microphone 230, a speaker 240, and speakers 250.

The mobile computer base 210 is coupled to the mobile computer display 220, speaker 240, and speakers 250. Mobile computer display 220 is coupled to microphone 230. The microphone 230 may be mounted to the top of the mobile computer display 220. The microphone 230 may be facing in a direction away from a user of the mobile computer. Noise in the computing working environment 200 is captured by microphone 230. A processor in the mobile computer base 210 may generate a noise cancellation waveform from the captured noise waveform. The mobile computer display may enable a user to adjust the algorithm used to generate the noise cancellation waveform. The noise cancellation waveform may be communicated to the computing working environment through speaker 240 and speakers 250. Speakers 250 may be surround sound speakers.

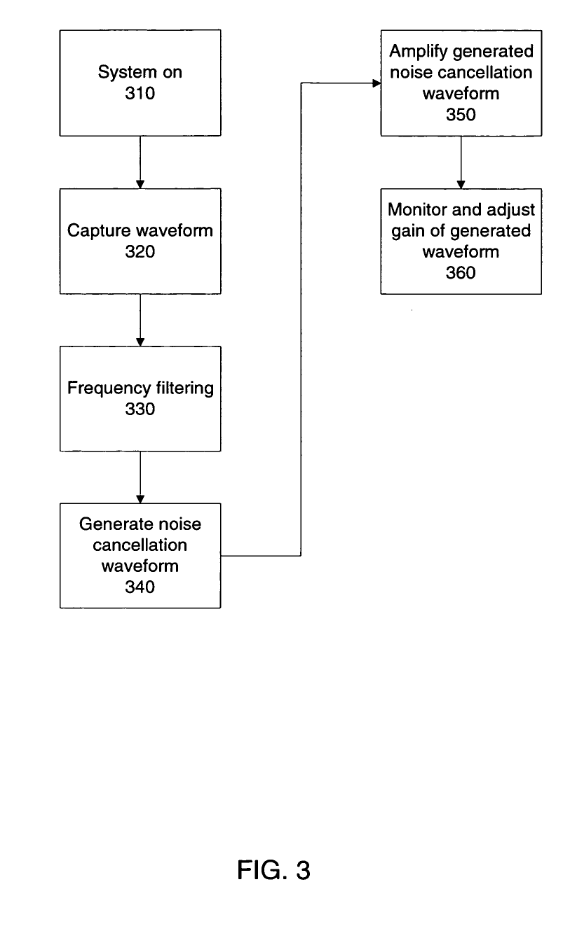

FIG. 3 depicts an embodiment of a flowchart for generating a noise cancellation waveform. A noise cancellation device is turned on in operation 310. The system then captures audio waveforms in the working environment in operation 320. An audio waveform may be captured using a microphone in the device. Next, the device may filter the frequency of the captured waveform in operation 330. The frequency filtering range may be adjusted. Noise level typically depends on a person's hearing range. Thus, depending on the user's hearing range, the user may choose to only cancel noise in certain frequency ranges. Moreover, the user may wish to block out certain noises while allowing others to be heard. For example, at the airport, a user may wish to generate a waveform to cancel the noise created by airport travelers, but may wish to hear airline announcements made over an intercom.

Based on the frequency filtering settings, the device generates a noise cancellation waveform in operation 340. The noise cancellation waveform may be generated by a processor that is part of the noise cancellation device. The processor may be a central processing unit or a digital signal processor. An embodiment of an algorithm for generating a noise cancellation waveform will be described in further detail below.

After the noise cancellation waveform is generated, the noise cancellation waveform is amplified in operation 350. The amplification may be performed by a codec or an amplification circuitry. The noise cancellation waveform is communicated to the working environment through a speaker. The gain of the noise cancellation waveform is monitored and adjusted in operation 360 to ensure that the noise cancellation waveform does not exceed the capabilities of the speaker. An automatic gain control circuitry may be used to clip the gain of the noise cancellation waveform at a predefined level.

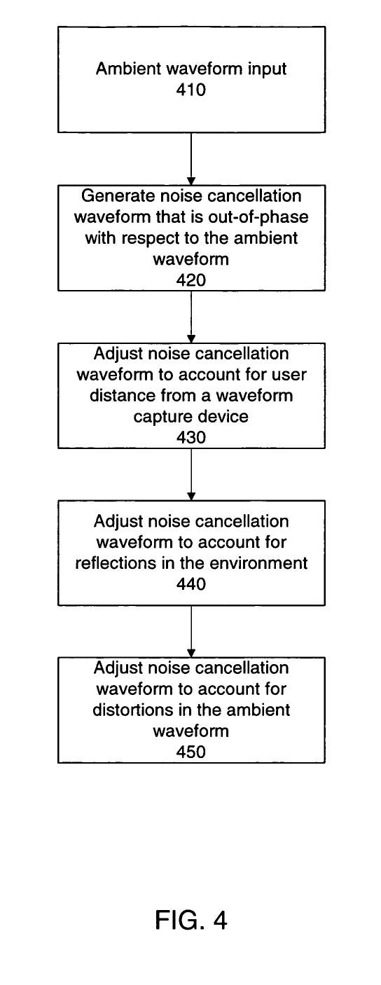

FIG. 4 depicts an embodiment of a flowchart of a noise generation algorithm. An ambient waveform from a working environment is received as an input in operation 410. A noise cancellation waveform is generated in operation 420. The noise cancellation waveform may be 180 degrees out-of-phase with respect to the ambient waveform. The amplitude and the period of the waveform, however, are approximately equal to the ambient waveform.

The noise cancellation waveform may then be adjusted in operation 430 to account for a user's distance from the waveform capture device. Because the user's ear may be a far distance from the waveform capture device, the waveform captured device may receive a noise waveform before or after the user depending on the location of the waveform capture device, the location of the user, and the location of the noise source. Thus, to account for the skew defined by the time difference between when the noise waveform is received by the user and when the noise waveform is received by the waveform capture device, the noise cancellation waveform may be adjusted accordingly. For one embodiment of the invention, the noise cancellation waveform may be delayed by a time period that it would take a noise cancellation waveform to travel the distance between the user and the waveform capture device. The user may input the delay parameter by setting his distance from the waveform capture device. Alternative, a processor may assume that the user is approximately one to five feet from the waveform capture device.

In operation 440, the noise cancellation waveform may be adjusted to account for reflections in the working environment. Noise waveforms in the working environment may reflect off walls or other elements of the working environment. Different materials reflect or absorb a different amount of noise. For example, aluminum blinds tend to reflect noise waveforms better than velvet curtains. Thus, even if a noise source is loud, the noise may reflect softly off a material that dampens the noise.

For one embodiment of the invention, a user may select from a list of features that may exist in a working environment. The list of features may include the size of the working environment, the material of the walls, and the fixtures in the room. Depending on the features selected, a lookup table may be used to select a reflective index. The reflective index may then be used to adjust the amplitude or the period of the noise cancellation waveform to cancel the reflection noise.

In operation 450, the noise cancellation waveform may be adjusted to account for distortions in the ambient waveform. Similar to reflection, one material may distort a noise waveform in a different manner than a second material. Noise distortion is caused by resonance or vibration. Each feature in the list of features used in deriving a reflective index may also be pre-characterized for its distortion properties. Thus, distortion variables will be set depending on the features, as selected by the user, that exist in the working environment. After adjusting for the delay parameter in operation 430, the reflective index in operation 440, and the distortion variables in operation 450, the noise cancellation waveform is communicated to the working environment.

Although the algorithm of FIG. 4 adjusts for the delay parameter before adjusting for the reflective index and the distortion variables, the order of the operations may be switched. For another embodiment of the invention, the algorithm may adjust for the reflective index before adjusting for the delay parameter and the distortion variables. For yet another embodiment of the invention, the algorithm may adjust the distortion variables before adjusting for the delay parameter and the reflective index.

Embodiments of the present invention may be implemented in hardware or software, or a combination of both. However, preferably, embodiments of the invention may be implemented in computer programs executing on programmable computer systems each comprising at least one processor, a data storage system (including volatile and non-volatile memory and/or storage elements), at least one input device, and at least one output device. Program code may be applied to input data to perform the functions described herein and generate output information. The output information may be applied to one or more output devices, in known fashion.

Each program may be implemented in a high level procedural or object oriented programming language to communicate with the computer system. However, the programs may be implemented in assembly or machine language, if desired. In any case, the language may be a compiled or interpreted language.

Each such computer program may be stored on a storage media or device (e.g., hard disk drive, floppy disk drive, read only memory (ROM), CD-ROM device, flash memory device, digital versatile disk (DVD), or other storage device) readable by a general or special purpose programmable computer system, for configuring and operating the computer system when the storage media or device is read by the computer system to perform the procedures described herein. Embodiments of the invention may also be considered to be implemented as a machine-readable storage medium, configured for use with a computer system, where the storage medium so configured causes the computer system to operate in a specific and predefined manner to perform the functions described herein.

In the foregoing specification the invention has been described with reference to specific exemplary embodiments thereof. It will, however, be evident that various modification and changes may be made thereto without departure from the broader spirit and scope of the invention as set forth in the appended claims. The specification and drawings are, accordingly, to be regarded in an illustrative rather than restrictive sense.

1. A method, comprising:
capturing a first waveform from a working environment with a microphone;
generating a second waveform to cancel the first waveform;
adjusting the second waveform based on a delay parameter that accounts for the distance between a user and the microphone; and
communicating the second waveform through a speaker.

2. The method of claim 1, further comprising:
monitoring and adjusting the gain of the second waveform communicated through the speaker, wherein the gain is clipped at a predetermined level.

3. The method of claim 1, further comprising:
selecting a reflective index based on features of the working environment, wherein the reflective index is a factor in generating the second waveform.

4. The method of claim 3, wherein the reflective index is obtained via a lookup table.

5. The method of claim 1, further comprising:
calculating a distortion variable, wherein the distortion variable is used to adjust the second waveform.

6. A computer system, comprising:
a processor;
a transmit audio circuitry coupled to the processor to amplify a noise cancellation signal;
a speaker coupled to the transmit audio circuitry to transmit the noise signal cancellation signal, wherein the speaker is mounted to a display facing away from a user of the computer system; and
a user interface coupled to the processor, wherein the user interface allows the user of the computer system to adjust variables used to generate the noise cancellation signal.

7. The computer system of claim 6, further comprising:
a memory coupled to the processor, wherein the memory comprises an algorithm to generate the noise cancellation signal.

8. The computer system of claim 6, further comprising:
a microphone coupled to the processor, wherein the microphone samples ambient noise.

9. The computer system of claim 8, further comprising:
a receive audio circuitry to the microphone, wherein the receive audio circuitry converts an analog signal to a digital signal.

10. The computer system of claim 6, wherein the transmit audio circuitry converts a digital signal to an analog signal.

11. The computer system of claim 6, wherein the computer system is a mobile computer system.

12. The computer system of claim 6, wherein the computer system is a desktop computer system.

13. An electronic device, comprising:
means for compensating for a delay in transmitting a noise cancellation signal to a user;
means for setting a reflective index; and
means for calibrating a signal distortion.

14. The electronic device of claim 13, further comprising:
means for capturing noise.

15. The electronic device of claim 13, further comprising:
means for communicating the noise cancellation signal.

16. The electronic device of claim 13, further comprising:
means for adjusting a frequency filter.

17. The electronic device of claim 13, further comprising:
means for limiting the gain of the noise cancellation signal.

18. An article comprising a machine readable medium having a plurality of machine readable instructions, wherein when the instructions are executed by a processor, the instructions cause a system to:
capture a noise in a working environment;
generate a noise cancellation signal based on a list of user selected elements of the working environment; and
transmit the noise cancellation signal through a speaker to offset the noise.

19. The article comprising the machine readable medium of claim 18, the instructions further cause the system to:
adjust the noise cancellation signal for distortions.

20. The article comprising the machine readable medium of claim 18, the instructions further cause the system to:
adjust the noise cancellation signal for a delay in transmitting the noise cancellation signal to a user.
